(19) United States
(12) Patent Application Publication
(10) Pub. No.: US 2020/0321091 A1
(43) Pub. Date: Oct. 8, 2020

(54) SYSTEM AND METHOD FOR PREDICTION OF MEDICAL TREATMENT EFFECT

(71) Applicant: OPTIMATA LTD., Tel Aviv (IL)

(72) Inventors: Eldad TAUB, Modiin-Maccabim-Reut (IL); Yuri KOGAN, Kiryat Ono (IL); Moran ELISHMERENI-LANDAU, Jerusalem (IL); Samuel SHANNON, Karmei Yosef (IL); Reuven EITAN, Giv'at Shmuel (IL)

(21) Appl. No.: 16/758,187

(22) PCT Filed: Oct. 24, 2018
(86) PCT No.: PCT/IL2018/051135

§ 371 Date: Apr. 22, 2020

(30) Foreign Application Priority Data
Oct. 25, 2017 (IL) 255255

(51) Int. Cl. G16H 20/00; G16H 50/30; G16H 50/50

(52) U.S. Cl. CPC G16H 20/00; G16H 50/50; G16H 50/30

(57) ABSTRACT

Systems and methods, for use during treatment of an individual having a certain disease and undergoing treatment under a specific line of treatment, are presented. The system comprises: a data input utility for receiving input data of the individual, the input data comprising two or more measured values of at least one medical parameter being measured at two or more respective time points, and comprising at least one in-treatment measured value measured since onset of the treatment under the specific line of treatment; a data processing utility for utilizing the input data and processing a disease progression model, corresponding to the certain disease, and determining from the measured values one or more disease stage indicator values, and processing the one or more disease stage indicator values to generate output data indicative of disease progression occurring within a predetermined treatment period; and a data output utility for outputting the output data thereby enabling a user of the system to decide about the course of treatment.

### TECHNOLOGICAL FIELD

This present invention is in the field of personalized medicine and relates to methods and systems for prediction of progression of disease.

### BACKGROUND

Personalized medicine approach has become attractive and important. Personalized medicine is known as providing a patient with “the right drug at the right dose at the right time”, thus it concerns tailoring of medical treatment to the individual characteristics, needs, and preferences of a patient during all stages of care, including prevention, diagnosis, treatment, and follow-up.

In general, a physician selects a treatment protocol/plan for a specific disease based on a number of considerations. According to these considerations, when a physician prescribes a specific treatment protocol for treating a disease in a specific patient, he/she may consider, inter alia, a plurality of treatment protocols, statistical data about the effect of the treatment protocols on previously treated patients, medical data of the specific patient including the current disease stage, disease progression data since diagnosis, as well as patient's age, general health, background illnesses, etc.

Practically, these considerations are usually based on statistics from clinical trials, and on the physician's subjective knowledge and experience for selecting one or more of the known treatment protocols for treating a specific patient having a certain disease. Some of the techniques for choosing the treatment protocol is described in WO15118529 assigned to the assignee of this patent application.

In the specific case of cancer therapy, the treatment includes one or more successive lines of therapy for a given type and stage of cancer, where each line includes a plurality of treatment protocols. Based on his/her experience and knowledge, the doctor chooses a treatment protocol from the several protocols included in the first line of treatment to start with. The patient undergoes treatment which extends over one or more treatment sessions within the specific line. The treatment session typically lasts for a predetermined time period, e.g. two to four months or as the doctor decides, and the patient undergoes periodical assessments of the disease state after each treatment session (every two-four months, or as the doctor determines) to evaluate treatment efficacy by examining the patient's response and identifying existence or lack of disease progression. Once disease progression is identified, the doctor stops the applied treatment protocol and considers which treatment protocol among the several treatment protocols included in the next line of treatment to proceed with, and so on.

Disease progression, such as in solid cancer, is assessed currently based on recognized progression criteria termed RECIST (Response Evaluation Criteria In Solid Tumors), currently in its 1.1 version, which is a set of published rules that define when cancer status improves (“complete response” or “partial response”), remains the same (“stable disease”) or worsens (“progression”) during therapy. One of the rules in the current RECIST criteria involves measuring the tumor burden, by estimating the sum of the longest diameters (SLD) of target lesions, and comparing it to the smallest SLD identified in the treated patient at the baseline or during the treatment. Roughly, the disease is said to have progressed if the SLD of target lesions increased significantly, if there is unequivocal progression in “non-target” lesions, or if new metastases occurred.

Another hint as to the tumor status in a patient is obtained by measuring the level(s) of “tumor marker(s)” in the body. Tumor markers are substances, e.g. proteins or small molecules that are produced by cancer cells or by other cells of the body in response to cancer or certain benign (noncancerous) conditions. Tumor markers are generally produced at higher levels in cancerous conditions, and can be found in the blood, urine, stool, tumor tissue, or other tissues or bodily fluids of patients. Therefore, tumor markers can be considered as indicators of the current disease severity in cancer, particularly at the specific time they are collected. For example, the prostate-specific antigen (PSA) level in blood is used as a surrogate tumor marker of current tumor burden in late-stage prostate cancer (PC). Similarly, carcinoembryonic antigen (CEA) is used for monitoring metastatic colorectal cancer (CC) during therapy, and persistently rising values above a certain value may suggest progressive disease.

### GENERAL DESCRIPTION

The present invention provides a novel approach for assisting a physician in treatment/therapy decision-making, inter alia this approach provides the physician with a powerful tool for making the right decision at the right timing, thus serving in increasing overall survival and quality of life for cancer patients, and specifically advanced late-stage cancer patients.

In particular, the invention provides a physician with a novel system that objectively detects and/or predicts progression of disease occurring already at, or after, a predetermined time (being for example proximate/imminent/immediate/subsequent progression), under any specific ongoing treatment line for any given patient, before the actual clinical manifestation of the progression. The prediction of disease progression occurrence alerts the physician and enables him/her to act in one of the following: updating the ongoing treatment, switching to a different treatment and/or subsequent line of treatment, or inviting the patient for an early assessment before deciding on the future treatment. Specifically, the system may be configured to generate as an output (e.g., in the form of a recommendation to the physician) one of the above-mentioned three options. By doing this, the invention provides the individual patient with a more efficacious treatment plan, thereby prolonging life expectancy.

The need for a novel approach is earnestly solicited in view of the lack of methods or technologies in the field for objectively predicting the future clinical outcome of treatment before the clinical outcome is a clinically verified fact (by conventional assessment of disease progression at defined periods). The matching of effective therapeutic protocols for patients with distinct characteristics is still far from optimal, causing many patients to still be treated with drugs that are in hindsight found ineffective for them. It is also a well-known fact that many therapeutics become less effective as the time passes, because the disease (even if initially is responsive to treatment) ultimately develops resistance against the treatment. Unfortunately, when the physician discovers this, it might be too late to rescue the patient or at least prolong his or her survival.

When considering tumor markers for example, there is no consensus on the use of tumor marker(s) (other than PSA and CEA) for monitoring the therapeutic effect of drugs and indicating disease status in any other solid cancer indication. For example, no single marker is elevated in a large proportion of advanced lung cancer (LC) patients to enable its exclusive use for monitoring disease progression. Similarly, in advanced breast cancer (BC), the tumor markers CEA, CA15-3, and CA27.29 are used only as adjunctive assessments together with imaging and physical examination for monitoring patients, and are not recommended for being used alone. Moreover, the above tumor markers, if reliable, are used as potential indicators of the disease severity at the specific time they are collected, and not as predictors of the disease state at a specific time point in the future. Presently, the guidelines on the use of tumor markers (e.g. of the American Society of Clinical Oncology, or of the European Group on Tumor Markers, and others) for monitoring treatment and detecting or predicting progression are vague and not solidified, and the evidence from different clinical studies and diverse patient populations on their application remains conflicting. Therefore, the testing of tumor markers in advanced cancer patients is mostly not mandatory and not binding, and in most cases it is still not applied for directing therapy.

At the periodical disease assessment, the physician examines the disease state by the RECIST criteria calculations, but also factors in additional considerations (including tumor marker levels, symptomatic state of the patient, and a multitude of other medical test results) in order to decide whether disease progression has occurred. The multiplicity of factors under consideration, and the lack of guidelines on the accurate interpretation of these factors together, can be at times confusing, painting a blurry picture of the disease's state, and complicating the decision-making process. Further, the consideration itself is frequently subjective and qualitative, rather than objective and quantitative. In other words, it heavily depends on the doctor's experience and subjective thinking Thus, the biomedical community still needs reliable tools for systematic personalized decision-making regarding effective treatment planning in cancer patients, specifically those with advanced metastatic cancer, by more accurate diagnosis of existing, as well as prediction of future-occurring, disease progression and response to therapy.

The present invention meets this need by providing a novel technique for detection of already ongoing or prediction of future-occurring disease progression in any given patient, while undergoing any specific treatment under any treatment line, based on processing of input data including one or more clinical parameter(s), specifically tumor marker(s) value(s).

For clarity, it should be noted that in this application the following terms have the following meanings, where some are given interpretation in accordance with the code of practice in the field. However, it should be understood that the mentioned meanings are by no means limiting, such that if the following terms or their meanings are changed according to the code of practice, then the invention can be adapted accordingly.

The term “treatment sequence”, or “sequence of treatment” of a given type and stage of disease (such as cancer), refers to the full treatment which a specific patient undergoes for treating his/her illness. It should be noted that the full treatment sequence (e.g. the multiple sequential treatment protocols that the patient is treated with, including the regimens and the number of cycles) is not known beforehand and its composition depends on how the patient's disease reacts to the treatment.

The sequence of treatment of a given type and stage of disease (e.g. cancer) includes a series of one or more consecutive “lines of treatment” (or “treatment lines”, “lines of therapy”), and the actual treatment of a patient in each treatment line includes typically one “treatment protocol” selected by the treating physician out of several treatment protocols which are recommended and approved for that line, where each treatment protocol consists of one or more drugs of respective doses. Accordingly, the treatment sequence starts with a treatment protocol under the first line of treatment, and, if needed, proceeds with another treatment protocol under the second line of treatment, etc.

Treatment within a specific treatment protocol in a given line extends over one or more “treatment cycles”, i.e. courses of treatment that are repeated on a regular schedule with periods of rest in between. Each cycle lasts for a predetermined period, typically two to four weeks, according to the Standard of Care. The “treatment cycle” is thus typically given multiple times within a specific treatment protocol.

Monitoring of the response to the treatment is done periodically at predetermined time intervals, typically every two to four months, e.g. every three months. However, the treating doctor may decide to perform assessment of the disease state prior to, or after, the predetermined time interval.

The term “treatment session”, as used herein, means a time interval between two successive evaluations (monitoring) of the disease state of an individual, which is either a predetermined typical interval, or an altered time interval as decided by the doctor. As appreciated, during one treatment session (lasting three months for example), the patient is treated with a specific treatment protocol over one or more treatment cycles.

As described, the sequence of treatment includes treating the patient with one or more consecutive lines of treatment, each line of treatment includes one treatment protocol (chosen by the physician from several possible treatment protocols under the specific line of treatment) to treat the patient with over one or more treatment sessions, each treatment session includes applying one or more treatment cycles of the specific treatment protocol. Switching to a subsequent line of treatment occurs after detecting progression of disease at the periodical assessments carried out typically at the end of each treatment session.

The term “medical data”, as used herein, refers to one or more medical parameter(s) value(s) collected at one or more time points in a specific individual undergoing treatment. The medical parameter(s) is/are indicative of the disease state, e.g. its severity, at the specific time it/they is/are measured. If more than one disease-state—indicating medical parameter is measured, it is either the singular effect of each indicative medical parameter or the collective effect of one or more groups of the indicative medical parameters that indicate the disease state/stage. In the specific case of cancer, the disease-state medical parameter(s) can be tumor marker(s) measured/evaluated by the treating physician in assessment of different cancer indications.

Optionally, in some embodiments, the medical data may include further data being indicative of the individual's medical state, such as medical history; patient characteristics (e.g. age, weight, height, gender, race, etc.); disease-related clinical data, e.g. pathology reviews; histologic subtype and immunohistochemistry (IHC); medical imaging data; blood counts (CBC); biochemistry profile; hormone profile and markers of inflammation; genetic and molecular diagnostic tests, e.g. mutation in one or more genes, one or more amplification in one or more copies, genetic recombination, partial or complete genetic sequencing; physical examination, and vitals.

The so-called “baseline medical data” includes medical data (as defined above) collected from the treated individual just before starting the treatment line under question, i.e. the treatment line that its efficacy is evaluated (e.g., at the periodical assessment(s)). In case the treatment line under question is not the first line applied, then the baseline medical data includes medical data collected after finishing treatment with the last treatment line and before starting the treatment line under question. The so-called “in-treatment medical data” or “current medical data” includes medical data (as defined above) obtained on the treated individual after starting the treatment line under question/evaluation.

Further, it should be noted that the definition of “disease progression” is made in accordance with the code of practice in the field, based on known criteria, e.g. according to the RECIST criteria, ver. 1.1, or any newer version.

The term “best medical disease state” defines the best medical condition of the individual with regard to the disease progression, as recognized in the specific field or disease. For example, the “nadir” criterion as recognized in the RECIST criteria.

To this end, the invention utilizes a system that employs computational and/or statistical and/or machine learning technique(s) (hardware or software product, or a combination thereof) for determining disease progression during treatment under any specific treatment line (i.e. in-treatment), for a specific individual patient who is already being treated under the specific treatment line. The system receives input data comprising medical data of the treated patient collected after starting the current treatment line (i.e. current, in-treatment medical data). The system analyses the input data, taking into account the best medical disease state recorded just before and after the onset of the current treatment line, and generates output data indicative of the efficacy of the current treatment line (e.g., in terms of disease progression). For example, the output can indicate whether or not the treatment is efficacious, y/n, in terms of disease progression at a predetermined future time point, e.g. at the end of the current treatment session, or at the immediate time of the application of the technique of the invention.

Therefore, the present invention is intended to be used for assessment of progression after the patient has already started treatment under a specific treatment line, given the patient's current (in-treatment and/or baseline) medical data. Upon detecting or predicting inefficacy of the currently applied treatment line, the physician will be able to either update the ongoing treatment protocol, switch to a treatment protocol included in the next line of treatment, or invite the patient for an early disease state assessment. The physician may receive, within the system output, a recommendation of the system as to which option of the above is best to proceed with, according to the specific circumstances.

According to the present invention, a disease-specific progression model (algorithm) is achieved by utilizing a set of computational/statistical/machine learning method(s) (e.g. neural networks, classification trees, regressions) that evaluate the probability of progression within a specified time period, as a function of the input data. These disease-specific models are constructed by employing advanced techniques of machine learning which are trained using training data set(s) that include large number of patients with the same disease/indication and who were treated by a specific treatment line (including one or more treatment protocols, and not necessarily identical to the treatment line under question) applied for that disease. For example, one or more features (e.g. statistical) extracted from the longitudinal dynamics of one or more tumor markers, measured during the treatment line under question, can be used as an input to these models/algorithms.

Thus, according to a broad aspect of the present invention, there is provided a system for use during treatment of an individual having a certain disease and undergoing treatment under a specific line of treatment for the certain disease, the system comprising:

a data input utility configured and operable to receive medical input data of the individual, the medical input data comprising two or more measured values of at least one medical parameter being measured at two or more respective time points, said two or more measured values of the at least one medical parameter comprising at least one in-treatment measured value of the at least one medical parameter being measured since onset of the treatment under the specific line of treatment;

a data processing utility configured and operable for utilizing said medical input data of the individual and processing a disease progression model, corresponding to the certain disease, and determining from said measured values of the one or more medical parameters one or more disease stage indicator values, and processing the one or more disease stage indicator values to generate output data indicative of disease progression occurring within a predetermined treatment period; and

a data output utility configured and operable for outputting said output data, thereby enabling a user of the system to perform one of the following (i) continue with the same treatment under the specific line of treatment (ii) update the treatment under the specific line of treatment, (iii) switch to treatment under a next line of treatment, or (iv) invite the individual for an early disease state assessment.

According to another broad aspect of the present invention, there is provided a method for use during treatment of an individual having a certain disease and undergoing treatment under a specific line of treatment for the certain disease, the method comprising:

providing input data of a specific individual comprising medical data comprising two or more measured values of at least one medical parameter being measured at two or more respective time points, said two or more measured values of the at least one medical parameter comprising at least one in-treatment measured value of the at least one medical parameter being measured since onset of the treatment under the specific line of treatment;

providing data indicative of a disease progression model corresponding to the disease;

utilizing said input data of the specific individual and data indicative of a disease progression model corresponding to the certain disease, and determining from said measured values of the at least one medical parameter one or more disease stage indicator values, and processing the one or more disease stage indicator values to generate output data indicative of prediction of future disease progression in said individual occurring within a predetermined time period; and

communicating said output data to a user, thereby enabling the user to perform one of the following: continue with the same treatment with the specific line of treatment, update the treatment under the specific line of treatment, switch to treatment under a next line of treatment, or invite the individual to an early disease state assessment.

According to some embodiments, the medical data further comprises baseline medical data comprising one or more measured values of the at least one medical parameter collected from the individual just before starting the treatment under the specific line of treatment.

According to some embodiments, the line of treatment is defined by one or more treatment protocols from which the user (of the system or the method, e.g., a treating doctor) chooses to treat the individual with.

According to some embodiments, the treatment under the specific line of treatment comprises one or more consecutive treatment sessions defining respective disease state assessment points carried out at the end of each treatment session, each treatment session lasting for a predetermined time interval, or as a treating doctor decides. The predetermined treatment period may be less than the predetermined time interval. The predetermined time interval may be between two to four months, or as the treating doctor decides.

According to some embodiments, the one or more disease stage indicator values are determined by extracting one or more features from longitudinal dynamics of the measured values of at least one medical parameter.

According to some embodiments, the output data is generated by comparing between the disease stage indicator values. Comparing between the disease stage indicator values may be carried out in accordance with a code of practice rules relating to the treatment of the certain disease.

According to some embodiments, the medical input data of the individual comprises two or more measured values of at least one additional medical parameter, thereby providing medical data about at least two medical parameters, each being measured at two or more respective time points, at least one of the disease stage indicator value(s) being determined by extracting at least one common feature from the measured values of the two or more medical parameters.

According to some embodiments, the method further comprises communicating with a database for accessing pre-stored reference data in the database, the reference data comprising data indicative of one or more of the following: one or more diseases, respective one or more code of practice rules, respective one or more lines of treatment for treating the one or more diseases, and respective one or more disease progression models.

According to some embodiments, the medical data further comprise one or more of the following: medical history; patient characteristics comprising age, weight, height, gender and race; disease-related clinical data; pathology reviews; histologic subtype;

immunohistochemistry (IHC); medical imaging data; blood counts (CBC);

biochemistry profile; hormone profile; markers of inflammation; genetic and molecular diagnostic tests; mutation in one or more genes; one or more amplification in one or more genetic copies; genetic recombination; partial or complete genetic sequencing; physical examination and vitals.

According to some embodiments, the output data comprises a yes/no answer with regard to disease progression occurring after the predetermined treatment period.

According to some embodiments, the medical parameter is indicative of the disease state at the time the medical parameter was measured.

According to some embodiments, the disease is cancer and the at least one medical parameter is a tumor marker.

According to some embodiments, the disease is lung cancer and the at least one medical parameter include CEA, CA19-9, CA-125 or CA15-3.

According to some embodiments, the treatment protocol comprises application of one or more drugs of one or more respective doses.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates one non-limiting example of a method for future prediction of disease state in accordance with the invention,

FIG. 2 illustrates one non-limiting example of a method for developing a disease-specific model of disease progression in accordance with the invention,

FIG. 3 illustrates a non-limiting example of a system for future prediction of disease state in accordance with the invention,

FIG. 4A illustrates disease state changes in time for an individual treated with three consecutive treatment lines according to the conventional practice, and

FIG. 4B illustrates disease state changes in time for the individual when treated with three consecutive treatment lines while utilizing the technique of the invention.

### DETAILED DESCRIPTION OF EMBODIMENTS

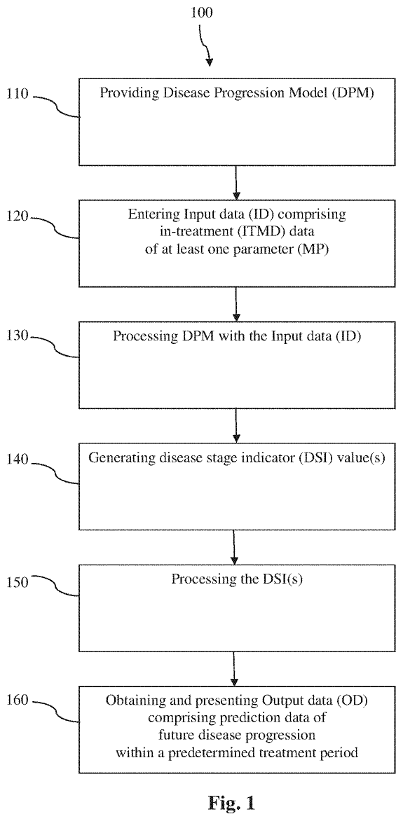

Reference is made to FIG. 1 illustrating schematically, by way of a flow diagram, a non-limiting example of a method 100 according to some embodiments of the present invention for estimating efficacy of a specific treatment line (TL) for an individual with a certain disease and undergoing treatment under the specific TL. It is noted that while the invention can generally be implemented and practiced with a variety of diseases (indications) and treatment methods, it is herein exemplified specifically with respect to cancer disease and cancer treatment methodologies. However, this should not be limiting the invention which is herein described in its broad meaning.

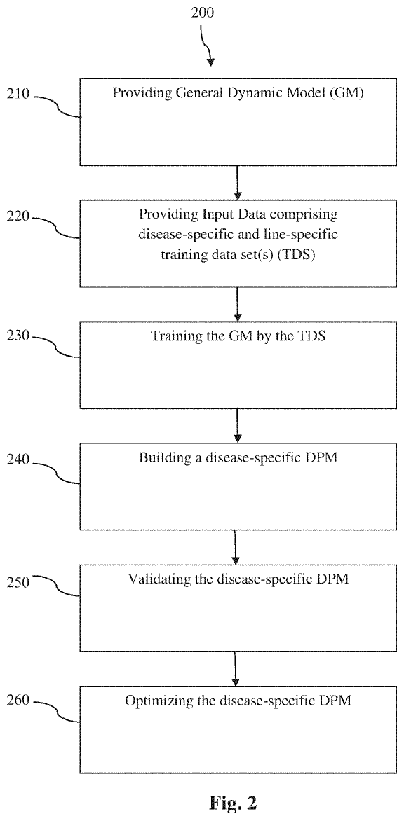

In step 110, a disease progression model (DPM) is provided. An example of a specific technique of development of the DPM is described herein further below with reference to FIG. 2. However, the development of the DPM is not necessarily part of method 100, and the DPM can be readily provided/accessed in a database. The DPM is disease-specific, meaning it is developed specifically for the certain disease for which the treatment efficacy is examined. For example, the DPM for use with a lung cancer patient is typically different from the DPM for use with a breast cancer patient. On the other hand, the DPM may or may not be specific to the treatment (e.g., a specific treatment protocol) applied. In other words, in at least some diseases, the DPM is the same for all the possible treatments (e.g., treatment protocols) of the disease, i.e. the DPM is treatment-independent. For example, as generally known, cancer therapy is composed of one or more consecutive treatment lines (TLs), and each TL includes one or more possible treatment protocols (TP) for treating the patient. Consequently, a patient is treated with a TP included in the first TL, and only if the treatment fails, such that a disease progression occurs under the TP of the first TL, the doctor switches to a TP from the second TL, and so on. As described also above, treating a patient under a specific TL (whether by a single or a variety of TPs belonging to the specific TL) may extend over one or more periodic treatment sessions, until a disease progression is diagnosed. Therefore, according to the invention, the DPM may be the same for all the lines of treatment applied in at least some of the diseases, or the DPM may be line-specific in some other diseases.

In step 120, input data (including patient-related data) is provided (e.g., being entered by a user (the doctor for example)), and in step 130, the DPM together with the input data are processed. The input data which is entered into and processed together with the DPM includes medical data including measured values (two or more) of at least one medical parameter (MP) being collected from the patient at two or more different times. Also, the input data may include the personal data of a specific patient (age, sex, etc.) and his/her disease and/or treatment history, if any.

Accordingly, the input data includes one or more measured values of the at least one MP being obtained after starting the treatment under the specific TL, this is called herein in-treatment medical data (ITMD). At least one measured value of ITMD of the MP is required. In case more than one treatment session has been carried out under the TL under examination, then some or all measured values of ITMD of the MP, measured since the beginning of the first treatment session under the TL, can be entered for processing with the DPM.

In some embodiments, the input data may further include one or more measured values of the MP obtained prior to starting the treatment with the TL under examination, this is a baseline medical data (BMD). It reflects the patient medical condition and the disease state, just before administration of drugs/medicine under the specific TL. Usually, one BMD value of the MP is sufficient. In case the TL under examination is a second or later line, the BMD is typically collected at the last assessment performed after finishing the last treatment session applied under the previous TL, or at a later time just before commencement of the treatment by the TL under examination.

Consequently, the two or more measured values of the MP, included in the input data, may be composed of either two or more ITMD measurements, or one or more ITMD measurements together with one or more BMD measurements.

As will be further exemplified below, in the specific case of cancer therapy, a treatment session may extend over two-four months on average, e.g. three months. The technique of the invention enables prediction of future disease state, e.g. at the end of the ongoing treatment session, after being for some time, e.g. a month or so, within the treatment session. While not necessarily measured at fixed spaced-apart time points, in other words not at a fixed frequency, the time between every two ITMD measurements can be shorter than the duration of the treatment session(s), thereby enabling predicting the future disease state also at the end of the first treatment session applied in the specific TL under examination.

It is noted that in various diseases, more than one MP can/should be measured. The different MPs can be measured concurrently at the same frequency (at a displaced time array) or at a different frequency, giving different number of measured values for each MP. As will be further described below, the prediction of the disease progression depends on the processing of some or all of MPs measured during the treatment by the TL (i.e., in-treatment data) and possibly also before the treatment by the TL (baseline data).

In some exemplary, non-limiting, embodiments, the ID may include, in addition to the BMD and ITMD, other individual data that may enhance the prediction of the future disease state. This may be disease-specific or line-specific or both, such that the other individual data may be useful or necessary in the prediction for some diseases or some TLs. The other individual data may include, for example, one or more of the following: medical history; patient characteristics (e.g. age, weight, height, gender, race, etc.); disease-related clinical data, e.g. pathology reviews; histologic subtype and immunohistochemistry (IHC); medical imaging data; blood counts (CBC); biochemistry profile; hormone profile and markers of inflammation; genetic and molecular diagnostic tests, e.g. mutation in one or more genes, one or more amplification in one or more copies, genetic recombination, partial or complete genetic sequencing; physical examination, and vitals. Further, in some non-limiting embodiments, the other individual data may be processed prior to entering the ITMD, and possibly the BMD, thereby generating a personalized DPM (PDPM) which is processed, in step 130, with the ITMD, and possibly also the BMD, to eventually generate the prediction data of the future disease state.

The processing of the DPM together with the ID, in step 130, yields generation of disease stage indicator(s) (DSI) in step 140. The DSI is a quantity that describes the disease stage, such as the disease severity, and it is defined in accordance with common rules pertaining to the specific medical field, for example the DSI can be calculated in accordance with a relevant code of practice. Specifically, in cancer therapy the code of practice can be that of the RECIST criteria, as mentioned above. Generally, one DSI calculated value can be indicative of several measured values of the MP. In some non-limiting embodiments, each measured value of the MP, being obtained at a specific time, yields a corresponding calculated value of the DSI. In some other non-limiting embodiments, a plurality of DSI calculated values are indicative of one measured value of the MP (e.g., a plurality of DSI values corresponding to a plurality of future time points). In some non-limiting embodiments, an array of measured values of the MP yields one-to-one, matching array (having same length, same number of elements) of calculated DSIs. In yet some other non-limiting embodiments, an array of measured values of the MP yields a different, non-matching array (of different length, different number of elements) of calculated DSIs. In some non-limiting embodiments, the transition between the measured value(s) of the MP and the calculated value(s) of the DSI fulfils the same mathematical function, whether linear or non-linear function. In case more than one MP is measured, then either each MP is processed to yield a corresponding DSI, or a plurality of MPs are processed together to yield a common representative DSI. The generation of the DSI value utilizes extracting one or more features of the longitudinal dynamics of the one or more MPs, either absolute feature(s) of each MP or relative feature(s) between two or more MPs. Generating a DSI value needs at least two measured MP values, the at least two measured MP values can be of the same MP, or one of a first MP and another of a second MP. When at least two measured values are obtained for each MP of a plurality of MPs, then it is possible to generate a DSI value for each MP alone, and it is possible to generate different DSIs for different combinations of the available MPs. Comparisons between the different DSIs can then be performed.

Non-limiting examples of features that can be extracted from the longitudinal dynamics of the MP, can be: time elapsed from nadir (minimal value of the marker measured since treatment start); absolute or relative difference between currently (or previously) measured marker value and the nadir; and/or current (absolute or relative) rate of change of marker level, estimated from slopes of the marker time course.

In step 150, the resulting DSI value(s) is/are processed in order to enable prediction of the future disease state, e.g. the disease progression at the end of the ongoing treatment session, and generate output data (OD) indicative thereof in step 160. In some embodiments, the processing of the DSI value(s) yields a future, hypothetical, DSI value at a predetermined future point, e.g. at the next disease assessment point such as at the end of the ongoing treatment session. Processing of the DSI calculated value(s), resulting from processing of each MP alone or from processing several MPs, can be achieved by comparing between them, or by comparing each of them to a predetermined threshold value, or by manipulating them via calculations or functions (e.g., adding, subtracting, multiplying, dividing, etc.), or by defining a disease trend or a disease profile over time. In the particular case of cancer therapy, as will be further described below, the processing of the DSI(s) can be as follows. The measured medical parameter(s) can be one or more tumor markers. Processing of the one or more tumor markers generates one or more DSIs that are then processed to generate a predicted DSI value at a predetermined future point, such as at the next disease assessment point at the end of the current, ongoing, treatment session. Then, the lowest (or highest) value among the calculated DSIs, including the future-predicted DSI, is identified and compared with the future-predicted DSI. The lowest (or highest) DSI value may represent the best (or the worst) medical disease state (BMDS) of the patient during the TL under examination. The comparison can be via subtraction or division for example. If the result is above a predetermined threshold value, as defined by the relevant code of practice, then future disease progression is predicted and vice versa.

It is noted that, at least in the specific case of cancer therapy, the method 100 is valid for a specific TL. In other words, the method 100 is applied on a specific TL and not across a plurality of TLs. Accordingly, it should be understood, inter alia, that the best medical disease state is redefined for every TL, i.e. the BMDS is a local value being relevant for the TL under examination and not a global value over the whole treatment sequence. Therefore, the BMDS value is reset for every TL in the course of the whole treatment sequence. The BDMS can be defined, for example, according to the RECIST criteria (current version is 1.1). However, it can be also defined according to the relevant code of practice as the case may be. In the following, some non-limiting examples for defining the BDMS are described. When a single tumor lesion is involved, the BDMS is typically related to the minimal value of the longest diameter of the lesion at any time point, whether before or after starting treatment. In case more than one lesion was identified before starting the treatment, the BDMS is defined according to the sum of longest diameters (SLD) at each time point. In yet other examples, in which the MP(s) is/are tumor marker(S), the BDMS is defined based on the calculated value(s) of the DSI, as described above. In some examples, the calculated DSI is correlated to the SLD parameter.

In step 160, the output data indicative of the disease progression at a point in the future (whether immediate/close or later/far future) is obtained and meaningfully presented to the user to thereby enable him/her to decide about the future treatment. The user will be able to decide whether to continue with the applied treatment protocol, especially if no progression is predicted, or change the treatment, for example by updating the treatment protocol under the same TL or switching to a subsequent TL, or invite the patient for an early disease assessment before making a final decision. The latter option can be useful, for example, if the prediction shows that the future disease state of the patient, represented by the future-predicted DSI value, will be worse than the lowest (or highest) DSI value although the difference is less than the threshold defined according to the relevant code of practice.

Reference is now made to FIG. 2 illustrating schematically, by way of a block diagram, a non-limiting example of a method 200 for the generation of the disease-specific disease progression model (DPM) according to some embodiments of the invention.

In step 210, a general, dynamic, model (GM) is provided. Generally, the GM can be a computational and/or statistical model composed of general functions, e.g. a model which can be adjusted for calculating probability of disease progression over time.

In step 220, input data including disease-specific and line-specific training data set(s) (TDS) is provided. The training data set(s) includes medical data of individuals who were diagnosed as having the specific disease. So, in order to build the DPM for lung cancer, for example, medical data of lung cancer patients is provided. In addition, the medical data of each individual should include medical data before starting treatment, i.e. start data, and data after finishing treatment, i.e. end data. The start and end data are entered as input to the GM. The medical data should be line-specific, in other words limited to a specific line of treatment, i.e. the start data reflects the medical condition of the individuals before starting the treatment by a specific TL and the end data reflects the medical condition of the individuals after finishing the treatment by the same TL. However, it should be noted that while the training data set(s) are limited to a specific TL, the developed DPM can be used with any TL (same or other) for treating the same disease, as the measured medical parameter is what matters. Therefore, the DPM can be developed using training dataset(s) including data about individuals treated for example under a first line of treatment, and used to predict disease progression in a patient treated with a second line of treatment, as long as the medical parameter(s) monitored in the patient is the same as the medical parameter(s) used in the training data set(s).

In some non-limiting embodiments, the medical start data includes measured values (raw data) of one or more medical parameters. In some non-limiting embodiments, the medical start data includes, solely or in addition to the raw data, calculated values obtained from the measured values of the medical parameter(s). These calculated values may be obtained by extracting features from the longitudinal dynamics of the medical parameter(s), such as the features described above with respect to step 140 of FIG. 1.

In step 230, the GM is trained using the training data set(s) and a disease-specific DPM is obtained in step 240. It should be noted, that the disease-specific and medical parameter-specific terms can be the same in some examples and can be used interchangeably, as each disease is defined by the medical parameters monitored in the patients. For example, for the invention, a specific cancer disease can be defined by the one or more tumor markers monitored in patients having the specific cancer disease. The training of the GM, to enable prediction of disease progression in a predefined period of time (yes/no), is carried out by using an advanced machine learning methodology, for example by correlating changes in measurements of the medical parameter(s) with foreseen clinical outcome (progression yes/no). The process of training the GM using the training data set(s) may provide functions describing relations between the medical data of the group of individuals and the output of the DPMs, these functions form integral part of or define the DPMs, enabling their personalization for a specific individual.

Optionally, in step 250, after developing the DPM, by training the GM with a TDS, the DPM's prediction accuracy can be validated, through a retrospective exploratory clinical study, by using an independent retrospective number of patient files (validation data set), per disease per treatment line.

Yet optionally, in step 260, the DPM can be continuously optimized during usage on every individual to make the DPM more robust.

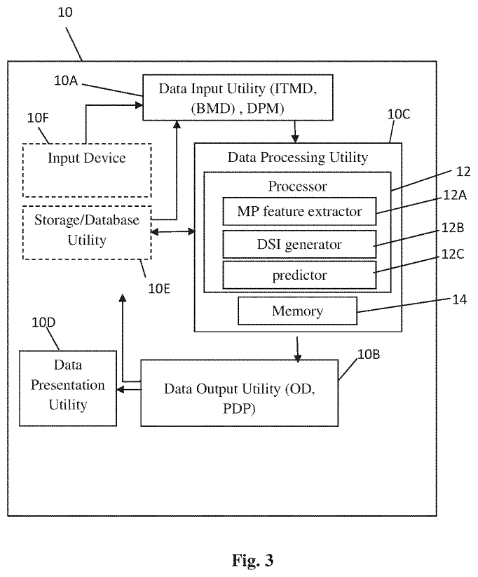

Reference is now made to FIG. 3 illustrating schematically, by way of a block diagram, one non-limiting example of a system 10 of the present invention for use during treatment for estimating efficacy of a specific treatment line for an individual having a certain disease and undergoing treatment by the specific treatment line. The system 10 can be used to execute the methods 100 and 200 described above. The system 10 is a computerized system, including inter alia such utilities (software and/or hardware) as data input and output utilities 10A, 10B, data processing utility 10C, and data presentation utility (e.g. display or speaker) 10D. The data processing utility 10C includes typically a processor 12 and a memory 14 (serving as transient as well non-transient memory to support the processor 12). Optionally, as shown by dashed lines, the system 10 can include input device 10F (e.g. keyboard, microphone, wireless link or a touch screen) and storage/database utility 10E (e.g. a memory device or a network/cloud based link), which alternatively can be external to the system 10 and communicating therebetween.

The system 10 receives via its input utility 10A certain input data as will be described further below, being provided by a user (e.g. a physician) via the input device 10F and/or by other connected external device (not shown) and/or by the storage/database utility 10E. Accordingly, the input utility 10A is appropriately configured to include user interface as well as a communication interface/utility (which are not specifically shown) for communication with external devices (e.g. input device, storage device, cloud storage, database, medical measurement device, server, etc.) via wires or wireless network signal transmission (e.g. RF, IR, acoustic, etc.). All these components and their operation are known per se and therefore need not be specifically described.

The input data utilized for the estimation of treatment efficacy and prediction of personal treatment effect includes, as described above with reference to method 100, in-treatment medical data (ITMD), and possibly also baseline medical data (BMD). As described, each of the in-treatment and baseline medical data includes one or more measured values of same at least one medical parameter being measured at respective one or more time points. In addition, the input data includes the DPM corresponding to the specific disease which the individual is diagnosed with.

The medical data of the specific individual (ITMD and optionally BMD) is entered by a user (e.g. a physician) to the data input utility, e.g. via the input device 10F, or from a storage device, such as storage utility 10E, where such data has been prepared/collected, or directly from connected one or more medical measurement/monitoring devices.

According to the invention, a plurality of DPMs, each per disease (indication), can be provided out-of-the-box to the system 10 to be used during treatment, for estimation of the treatment efficacy, e.g. by saving them in the storage utility 10E, whether it is internal or external to the system 10, such that any of them can be accessed and run (simulated), upon the user decision. Alternatively, in some embodiments, the system 10, i.e. its data processing utility 10C, is configured to execute method 200 and obtain or update the DPM, independently without interference from the user.

The prediction on progression of disease PDP, is generated by the data processing utility 10C, e.g. in accordance with the code of practice, e.g. the RECIST criteria version 1.1, as was described above in connection with method 100. The output data OD generated by the data processing utility 10C, including the PDP, is delivered by the data processing utility 10C to the output data utility 10B which conveys the output data to the user, via the data presentation utility 10D, in a meaningful clear manner, e.g. visual, or audible output. Accordingly, the data presentation utility 10D can include a display or a speaker or both. In some embodiments, the prediction of progression of disease PDP can be indicative of a yes/no answer, such that the user is simply informed, visually or audibly, whether a progression will occur or not, after a predetermined future treatment period, so that he can calculate his next step in the treatment. In some embodiments, the output data can include a probabilistic prediction providing chance of progression after a predetermined treatment period. In some embodiments, the output data can include a graph of the predicted disease progression as a function of time, enabling the user to evaluate the disease state at different future time points. For example, in the latter case, the user is given information enabling him to decide about continuation with the current treatment until a time point in the future being earlier than the conventional time point at which the current treatment session should have been finished. In yet some embodiments, the output data includes comparison between the predicted progression of disease under a plurality of different doses of the drugs included in the ongoing treatment protocol, thus helping the user in his/her decision about the subsequent treatment. In yet some embodiments, according to the processing of the input data and the DPM, the system 10 (by its data processing utility 10C or its data output utility 10B) is configured to generate output data that includes a direct recommendation for the treating doctor about the next step of treatment; the direct recommendation can be, for example, one of the following: update the ongoing treatment until the end of the current treatment session (either update the current treatment protocol (e.g. update the doses of one or more medicines), or change the treatment protocol to another treatment protocol included in the same treatment line, or a combination thereof (sequentially or concurrently)), move to the next line of treatment (with or without specific recommendation about one of the treatment protocols, included in the next TL, based on a simulation executed by the system), or invite the patient for an evaluation examination procedure (e.g. imaging) now and not wait until the next scheduled one.

The processor 12 is configured to process the input data and/or the DPM(s), in accordance with the method 100, in order to generate the output data enabling to estimate the treatment efficacy. As such, the processor 12 may include such modules as a MP feature extractor module 12A configured and operable to extract one or more features of the measured values of the one or more MPs, a DSI generator module 12B configured and operable to execute the steps 140 and 150 of method 100 to calculate the DSI(s) and process the DSI(s) to generate a common DSI indicative of the future prediction of the treatment efficacy, and a predictor module 12C configured and operable to generate the OD and PDP.

As described above, while not specifically illustrated, in some embodiments the data processing utility 10C is configured for developing and generating the DPM for each disease (indication) and each treatment line by utilizing the method 200.

The DPMs together with the input data can then be simulated in the data processing utility 10C with respect to each treatment protocol/line to thereby evaluate the effects of the treatment.

As described above, the present invention is particularly useful in usage with cancer patients and provides a powerful tool for use during treatment given to the patient. The invention utilizes the accepted tumor marker(s) monitored by the physicians community as being indicators of the cancer stage or severity. In some examples, the invention may utilize one or more tumor markers that are not necessarily monitored or recognized by the medical community as being indicators of the stage of a specific cancer or any of its underlying processes, either ultimately or in addition to recognized tumor marker(s). Non-limiting examples of the tumor marker(s) currently recognized and used for each disease are as shown in the following Table 1:

| | | | | Year first |
|---|---|---|---|---|
| | | | | approved or |
| Biomarker | Clinical use | Cancer type | Specimen | cleared |
| Pro2PSA | Discriminating cancer | Prostate | Serum | 2012 |
| | from benign disease | | | |
| ROMA (HE4 + CA-125) | Prediction of | Ovarian | Serum | 2011 |
| | malignancy | | | |
| OVA1 (multiple proteins) | Prediction of | Ovarian | Serum | 2009 |
| | malignancy | | | |
| HE4 | Monitoring recurrence | Ovarian | Serum | 2008 |
| | or progression of | | | |
| | disease | | | |
| Fibrin/fibrinogen | Monitoring | Colorectal | Serum | 2008 |
| degradation product (DR- | progression of disease | | | |
| 70) | | | | |
| AFP-L3% | Risk assessment for | Hepatocellular | Serum | 2005 |
| | development of disease | | | |
| Circulating Tumor Cells | Prediction of cancer | Breast | Whole | 2005 |
| (EpCAM, CD45, | progression and | | blood | |
| cytokeratins 8, 18+, 19+) | survival | | | |
| p63 protein | Aid in differential | Prostate | FFPE tissue | 2005 |
| | diagnosis | | | |
| c-Kit | Detection of tumors, | Gastrointestinal | FFPE tissue | 2004 |
| | aid in selection of | stromal tumors | | |
| | patients | | | |
| CA19-9 | Monitoring disease | Pancreatic | Serum, | 2002 |
| | status | | plasma | |
| Estrogen receptor (ER) | Prognosis, response to | Breast | FFPE tissue | 1999 |
| | therapy | | | |
| Progesterone receptor (PR) | Prognosis, response to | Breast | FFPE tissue | 1999 |
| | therapy | | | |
| HER-2/neu | Assessment for | Breast | FFPE tissue | 1998 |
| | therapy | | | |
| CA-125 | Monitoring disease | Ovarian | Serum, | 1997 |
| | progression, response | | plasma | |
| | to therapy | | | |
| CA15-3 | Monitoring disease | Breast | Serum, | 1997 |
| | response to therapy | | plasma | |
| CA27.29 | Monitoring disease | Breast | Serum | 1997 |
| | response to therapy | | | |
| Free PSA | Discriminating cancer | Prostate | Serum | 1997 |
| | from benign disease | | | |
| Thyroglobulin | Aid in monitoring | Thyroid | Serum, | 1997 |
| | | | plasma | |
| Nuclear Mitotic Apparatus | Diagnosis and | Bladder | Urine | 1996 |
| protein (NuMA, NMP22) | monitoring of disease | | | |
| Alpha-fetoprotein (AFP) ^{D} | Management of cancer | Testicular | Serum, | 1992 |
| | | | plasma, | |
| Total PSA | Prostate cancer | Prostate | Serum | 1986 |
| | diagnosis and monitoring | | | |
| Carcinoembryonic antigen | Aid in management and | Not specified | Serum, | 1985 |
| (CEA) | prognosis | | plasma | |
| Human hemoglobin (fecal | Detection of fecal occult | Colorectal | Feces | 1976 |
| occult blood) | blood (home use) | | | |

The invention provides a quantitative and objective approach instead of the qualitative and subjective approach used so far by the physicians. Accordingly, for each disease in the list, a DPM is built according to the invention, by training the GM with TDS of patients, the TDS include data of at least the tumor marker(s) included in the list.

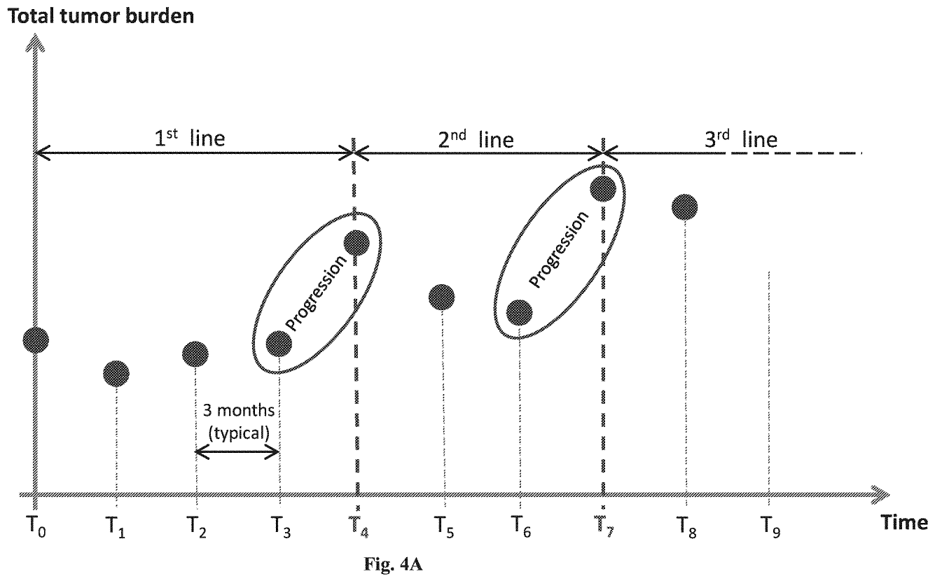

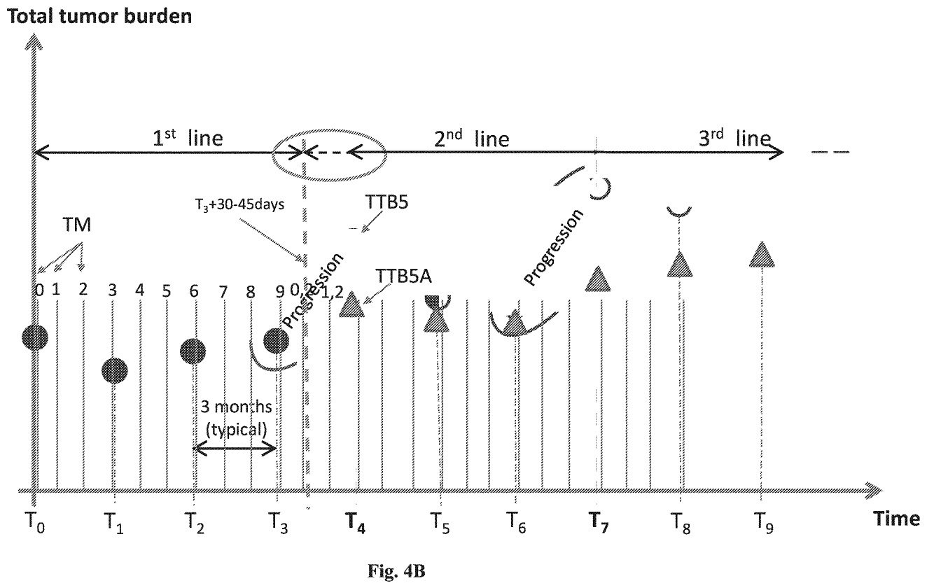

Reference is now made to FIGS. 4A-4B illustrating non-limiting exemplary embodiment of utilizing the invention in the prediction of treatment outcome and disease progression in a cancer patient.

FIG. 4A illustrates one non-limiting hypothetical example of a treatment sequence carried out in a specific individual along with the disease state of the individual according to the conventional practice, compared with FIG. 4B illustrating the disease state of the individual when the treatment is accompanied by efficacy estimation performed according to method 100 and/or by the system 10 according to the invention.

As shown, FIG. 4A includes a graph highlighting the individual's disease state on the Y-axis, in this example by the total tumor burden (which is defined by sum of longest diameters (SLD), non-target lesions and new lesions), as a function of time on the X-axis, as illustrated by time points T₀-T₉ which indicate disease assessment points along the treatment sequence. It should be understood that the Y-axis is not linear in that the total tumor burden does not necessarily increase or decrease linearly. For example, appearance of new lesions increases the total tumor burden by more than its actual addition to the SLD. As shown on the graph, in par with the code of practice, each time interval between successive measurements (treatment session) is, for example, three months. The individual is given treatment and his/her disease state is examined by the suitable means, e.g. by imaging, every three months. If a disease progression is identified, the physician changes the treatment by switching to the next TL and choosing a treatment protocol from the ones included in the next TL. As can be seen in this example, the graph includes three consecutive lines of treatment, where a first treatment protocol under the first line is given during four treatment sessions of three months each, a second treatment protocol under the second line is given during another three treatment sessions of three months each, and a third treatment protocol under the third line is given during another two treatment sessions of three months each. According to the code of practice, each treatment protocol belonging to a specific line of treatment is usually given to the individual until a progression of disease is identified. As shown, a progression event was identified at T₄, when a disease assessment was carried out after the fourth treatment session in which the individual was treated with a treatment protocol of the first treatment line. After moving to the second treatment line, with a second treatment protocol, there was a decline in the disease at the end of treatment sessions five and six, at T₅ and T₆, then another progression was identified during the seventh treatment session with the second treatment protocol of the second treatment line. A third treatment protocol, chosen from treatment protocols of the third line, was started during the eighth treatment session. As can be seen, this caused the patient to end up with a relatively high total tumor load, being suggestive of deterioration in his/her overall health and survival.

In contrast, as shown in FIG. 4B, by using the invention, the overall health and expected survival is improved for the individual. When using system 10 a while after starting each treatment session, e.g. after thirty-forty five days from each treatment session beginning, the disease is better controlled. In this example, the system 10 predicted no progression after starting each of the first three treatment sessions and the physician kept using the first treatment protocol. A while after beginning the fourth treatment session, still treating by the first treatment protocol, the system 10 predicts that treatment by the first treatment protocol is no longer effective. The physician switches to a second treatment protocol of a second treatment line, not waiting for the end of the fourth treatment session, thus resulting in better control of the disease with no progression identified at the end of treatment session 4, at T₄, as indicated by total tumor burden 5A (TTBSA) obtained at the fifth assessment point, instead of TTBS which would have been obtained should the treatment continues under the first line. Then, again, by continuing with using the system 10 a while, e.g. about thirty days, after the beginning of each treatment session, the system identifies, as shown, after a short while from starting the seventh treatment session with the second treatment protocol, that a progression of disease will be identified at the end of the seventh time interval T₇ if the treatment with the second treatment protocol continues. The physician again stops treatment with the second treatment protocol of the second line of treatment and switches to a third treatment protocol of a third treatment line, preventing the disease progression and resulting in a better overall control of the disease progression and increase in the overall survival time and quality of life for the treated individual. The actual TTBs from T₄ onwards, by using the invention, are indicated by the triangles, whereas the TTBs that would be obtained without the invention are indicated by the circles.

According to the invention, two or more values of one or more tumor markers (TM) values are measured throughout the treatment sequence, i.e. all the treatment sessions. The TM values measured since the start of each treatment line are used as the input to the DPM of the invention, in order to predict disease state (yes/no progression) after a predetermined future treatment time, e.g. after about sixty days or at the expected end of the ongoing treatment session. The values of the one or more TMs are measured with a predetermined frequency/pace that is relatively steady. If more than one TM is measured, it is not necessary that all the TMs are measured at the same time or at the same frequency/pace. Generally, the pace of the TM measurements is faster than the pace of the disease assessment points at the end of each treatment session (usually, two-four months). In the described non-limiting example, the TM value is measured roughly every month (thirty days or so), as shown by the lines 0, 1, 2 . . . , 9 indicating the number of measurements. So, before starting the first treatment session of the first line, TM₀ was measured forming baseline medical data for the first TL. Afterwards, the TM is measured every month or so, such that TM₁-TM₃ are measured during and at the end of the first treatment session, TM₄-TM₆ are measured during and at the end of the second treatment session, and so on. When the system 10 is used according to the invention at T₁+30 days for example, TM₀-TM₄ may be used as an input to the DPM to predict the disease state at T₂. In the described example, the invention predicts progression of the disease at T₃+30-45 days. The doctor has three options to proceed: update the ongoing treatment (e.g. update the ongoing treatment protocol or change to another treatment protocol included in the same treatment line), switch to the next line and not wait until the end of the ongoing treatment session, or invite the patient for an early assessment. The system 10 may be configured to output a recommendation of the next treatment step to the doctor. In the described example, the doctor switches to the second TL. Once the next TL starts, the TM measured values that form part of the input data to the DPM are those measured afterwards forming the in-treatment medical data, such as TM1,₂ shown on the graph. Possibly, the TM value measured just before the second TL, indicated TM0,₂, forms a baseline medical data and may be included in the input data. As can be understood from the above example, the TM values are reset at the start of each treatment line. Accordingly, as exemplified, using the invention helps the doctors in the planning of the treatment such that it is more effective in slowing the disease progression and improving the overall survival of the patients.

Non-limiting examples of experiments performed by the inventors using the technique of the present invention will be now described. The results are shown in the tables listed below. As will be appreciated, the results using the invention are compared with a hypothetical attempt to quantify the information provided in these markers by simple statistical tools (e.g. using receiver-operating-characteristic (ROC) analysis for quantifying the tumor markers predictive ability). A retrospective study on a group of 167 patients diagnosed with Non-Small Cell Lung Carcinoma (NSCLC) and treated with various therapies included in the first line of treatment was conducted by the inventors.

Table 2A summarizes the sensitivity and specificity of the five tumor markers examined (CEA, CA125, CA15.3, CA19.9 and NSE) when using the invention (results in rows 3 and 4) compared to applying basic statistical tools (e.g. ROC) on the same data, in a hypothetical study that is clinically practical (rows 1 and 2). As well-appreciated, both the sensitivity and specificity increase in all the above-mentioned tumor markers when using the invention. As currently used by known techniques, tumor markers carry weak signals. Conversely, by extracting one or more features from the longitudinal dynamics of each tumor marker, the invention boosts the weak signal of the tumor marker and transforms it into a strong indication of disease progression and treatment efficacy.

TABLE 2A

| | Tumor Marker | CEA | CA125 | CA15.3 | CA19.9 | NSE |
|---|---|---|---|---|---|---|
| As currently | Sensitivity (%) | 25.4 | 25.9 | 26.4 | 27.1 | 13.8 |
| used | Specificity (%) | 89.9 | 90.1 | 90.1 | 90.0 | 90.2 |
| With | Sensitivity (%) | 33.4 | 34.3 | 48.9 | 33.7 | 26.2 |
| invention | Specificity (%) | 90.1 | 91.6 | 91.3 | 91.0 | 91.9 |

Table 2B illustrates that combining and/or integrating one or more features of the longitudinal dynamics of a plurality of tumor markers may further boost the weak signals of the individual tumor markers, and may enhance the performance of the invention over the cases of the individual tumor markers. For example, by integrating features of the markers CAE, CA125 and CA15.3, using the invention, the sensitivity increases to 52.6%, while integrating features of all the five markers, using the invention, increases the sensitivity further up to 65.6%.

As also appreciated, as the patients have undergone treatment with various therapies and the sensitivity and specificity have increased for all tumor markers, regardless of the given therapy, the technique of the invention proves to be unaffected by the treatment type given, i.e. the technique of the invention is treatment-independent.

TABLE 2B

| | Tumor | CEA + CA125 + | CEA + CA125 + CA15.3 + |
|---|---|---|---|
| | Marker | CA15.3 | CA19.9 + NSE |
| With | Sensitivity | 52.6 | 65.6 |
| invention | (%) | | |
| | Specificity | 91.1 | 90.6 |
| | (%) | | |

Thus, the technique of the invention is robust, being treatment-independent and serving any patient of a specific disease being treated with any treatment protocol under any treatment line, as long as the DPM is developed on data of patients having the same disease and treated with any treatment protocol belonging to a certain TL.

1. A system for use during treatment of an individual having a certain disease and undergoing treatment under a specific line of treatment for the certain disease, the system comprising:
a data input utility configured and operable to receive medical input data of the individual, the medical input data comprising measured data of one or more medical parameters, the measured data of each one of the one or more medical parameters comprises two or more measured values of the medical parameter being measured at two or more respective time points, said two or more measured values of the medical parameter comprising at least one in-treatment measured value of the at least one medical parameter being measured since onset of the treatment under the specific line of treatment;
a data processing utility configured and operable for utilizing said medical input data of the individual and processing a disease-specific progression model, corresponding to the certain disease, and determining from said measured data of the one or more medical parameters one or more disease stage indicator values, and processing the one or more disease stage indicator values to generate output data indicative of disease progression occurring within a predetermined treatment period under the specific line of treatment; and
a data output utility configured and operable for outputting said output data, thereby enabling a user of the system to perform one of the following (i) continue with the same treatment under the specific line of treatment (ii) update the treatment under the specific line of treatment, (iii) switch to treatment under a next line of treatment, or (iv) invite the individual for an early disease state assessment.

2. The system according to claim 1, wherein said data processing utility is further configured and operable to generate the output data comprising a recommendation to the user for one of the following: continue with the same treatment under the specific line of treatment as previously planned, update the treatment under the specific line of treatment, switch to treatment under a next line of treatment, or invite the individual for an early disease state assessment.

3. The system according to claim 1, wherein said data input utility is configured and operable for receiving the medical input data further comprising baseline medical data comprising one or more measured values of said medical parameter collected from the individual just before starting the treatment under the specific line of treatment.

4. The system according to claim 1, wherein said line of treatment is defined by one or more treatment protocols from which the user chooses to treat the individual with, each of said one or more treatment protocols comprises application of one or more drugs of respective one or more doses.

5. The system according to claim 1, wherein said treatment under the specific line of treatment comprises one or more consecutive treatment sessions defining respective points of disease state assessment carried out at the end of each treatment session, each treatment session lasting for a predetermined time interval, or as a treating doctor decides.

6. The system according to claim 5, wherein said predetermined time interval is characterized by at least one of the following: a) it is longer than said predetermined treatment period, b) it is between two to four months, c) it is as the treating doctor decides.

7. (canceled)

8. The system according to claim 1, wherein said data processing utility is configured and operable to determine said one or more disease stage indicator values by performing at least one of the following: a) extracting one or more features from longitudinal dynamics two or more of the measured values of the medical parameter, b) when the medical input data comprises measured data of two or more medical parameters, extracting at least one common feature from the measured values of the two or more medical parameters.

9. The system according claim 1, wherein said data processing utility is configured and operable to generate said output data by comparing between said disease stage indicator values, based on code of practice rules relating to the treatment of the certain disease.

10-11. (canceled)

12. The system according to claim 1, further comprising a communication utility for communicating with a database for accessing pre-stored reference data comprising data indicative of one or more of the following: one or more diseases, respective one or more code of practice rules, respective one or more lines of treatment for treating the one or more diseases, and respective one or more disease-specific progression models.

13. The system according to claim 1, wherein said medical input data further comprise one or more of the following: medical history; patient characteristics comprising age, weight, height, gender and race; disease-related clinical data; pathology reviews; histologic subtype; immunohistochemistry (IHC); medical imaging data; blood counts (CBC); biochemistry profile; hormone profile; markers of inflammation; circulating tumor cells (CTCs); genetic and molecular diagnostic or prognostic tests; mutations in one or more genes; one or more amplifications in one or more genetic copies; genetic recombination; partial or complete genetic sequencing; physical examination and vitals.

14. (canceled)

15. The system according claim 1, wherein said at least one medical parameter is indicative of the disease state at the time the medical parameter was measured.

16. The system according to claim 1, wherein said disease is cancer and said at least one medical parameter are, respectively, one of the following: a cancer, a tumor marker b) lung cancer, at least one of CEA, CA19-9, CA-125 and CA15-3.

17-18. (canceled)

19. A method for use during treatment of an individual having a certain disease and undergoing treatment under a specific line of treatment for the certain disease, the method comprising:
providing input data of a specific individual comprising medical data comprising measured data of one or more medical parameters, the measured data of each one of the one or more medical parameters comprises two or more measured values of the medical parameter being measured at two or more respective time points, said two or more measured values of the medical parameter comprising at least one in-treatment measured value of the medical parameter being measured since onset of the treatment under the specific line of treatment;
providing data indicative of a disease-specific progression model corresponding to said disease;
utilizing said input data of the specific individual and data indicative of a disease-specific progression model corresponding to the certain disease, and determining from said measured data of the one or more medical parameter one or more disease stage indicator values, and processing the one or more disease stage indicator values to generate output data indicative of prediction of future disease progression in said individual occurring within a predetermined time period under the specific line of treatment; and
communicating said output data to a user, thereby enabling the user to perform one of the following: continue with the same treatment with the specific line of treatment, update the treatment under the specific line of treatment, switch to treatment under a next line of treatment, or invite the individual to an early disease state assessment.

20. The method according to claim 19, wherein said medical data further comprises baseline medical data comprising one or more measured values of said at least one medical parameter collected from the individual just before starting the treatment under the specific line of treatment.

21. The method according to claim 19, wherein said line of treatment is defined by one or more treatment protocols from which the user chooses to treat the individual with, each of said one or more treatment protocols comprises application of one or more drugs of respective one or more doses.

22. The method according to claim 19, wherein said treatment under the specific line of treatment comprises one or more consecutive treatment sessions defining respective points of disease state assessment carried out at the end of each treatment session, each treatment session lasting for a predetermined time interval, or as a treating doctor decides.

23. The method according to claim 22, wherein said predetermined time interval interval is characterized by at least one of the following: a) it is longer than said predetermined treatment period, b) it is between two to four months, c) it is as the treating doctor decides.

24. (canceled)

25. The method according to claim 19, wherein said one or more disease stage indicator values are determined by performing at least one of the following: a) extracting one or more features from longitudinal dynamics of the two or more measured values of the medical parameter, b) when the medical data comprises measured data of two or more medical parameters, extracting at least one common feature from the measured values of the two or more medical parameters.

26. The method according to claim 19, wherein said output data is generated by comparing between said disease stage indicator values, based on code of practice rules relating to the treatment of the certain disease.

27-28. (canceled)

29. The method according to claim 19, further comprising communicating with a database for accessing pre-stored reference data in the database, said reference data comprising data indicative of one or more of the following: one or more diseases, respective one or more code of practice rules, respective one or more lines of treatment for treating the one or more diseases, and respective one or more disease-specific progression models.

30. The method according to claim 19, wherein said medical input data further comprise one or more of the following: medical history; patient characteristics comprising age, weight, height, gender and race; disease-related clinical data; pathology reviews; histologic subtype; immunohistochemistry (IHC); medical imaging data; blood counts (CBC); biochemistry profile; hormone profile; markers of inflammation; genetic and molecular diagnostic tests; mutation in one or more genes; one or more amplification in one or more genetic copies; genetic recombination; partial or complete genetic sequencing; physical examination and vitals.

31. (canceled)

32. The method according to claim 19, wherein said medical parameter is indicative of the disease state at the time the medical parameter was measured.

33. The method according to claim 19, wherein said disease and said at least one medical parameter are respectively, one of the following: a cancer, a tumor marker b) lung cancer, at least one of CEA, CA19-9, CA-125 and CA15-3.

34-35. (canceled)
